(19) United States
(12) Patent Application Publication
(10) Pub. No.: US 2019/0151291 A1
(43) Pub. Date: May 23, 2019

(54) IFETROBAN TREATMENT OF PORTAL HYPERTENSION

(71) Applicant: Cumberland Pharmaceuticals Inc., Nashville, TN (US)

(72) Inventor: Leo PAVLIV, Cary, NC (US)

(21) Appl. No.: 16/258,606

(22) Filed: Jan. 27, 2019

Related U.S. Application Data
(63) Continuation of application No. 15/683,134, filed on Aug. 22, 2017, now abandoned.
(60) Provisional application No. 62/377,980, filed on Aug. 22, 2016.

(51) Int. Cl. A61K 31/422; A61K 9/00; A61K 9/20; A61K 47/02; A61K 9/48; A61K 9/08

(52) U.S. Cl. CPC A61K 31/422; A61K 9/0019; A61K 9/0053; A61K 9/20; A61K 9/48; A61K 9/4858; A61K 9/2059; A61K 9/08; A61K 47/02

(57) ABSTRACT

The present invention is directed to methods of treating and/or ameliorating portal hypertension by administration of a therapeutically effective amount of ifetroban or a pharmaceutically acceptable salt thereof.

### FIELD OF THE INVENTION

The present invention is related to the use of thromboxane A₂ receptor antagonists (e.g., ifetroban) in the treatment and/or prevention of portal hypertension in mammals, e.g., humans, and pharmaceutical compositions for the same comprising thromboxane A₂ receptor antagonists (e.g., ifetroban) in an effective amount to treat and/or prevent these diseases.

### BACKGROUND OF THE INVENTION

Cirrhosis is a potentially life-threatening condition that occurs when the liver is damaged by fibrotic scarring. Fibrosis can impair the flow of portal blood through the hepatic sinusoids and result in hypertension in the portal vasculature. One of the major complications from portal hypertension is the development of gastrointestinal varices. Bleeding due to varices specifically in the esophagus accounts for one third of the deaths related to portal hypertension. The median survival period in humans following diagnosis of cirrhosis is six years (La Vecchia 1994; Pagliaro 1994). Currently little effective therapy is commercially available for treatment of portal hypertension.

In the study of portal hypertension, there is key interest in mediators such as thromboxane A₂ (TxA₂), F2-isoprostanes, nitric oxide, and endothelin-1 as well as the thromboxane receptor (TPr) which has TxA₂ and F2-isoprostanes as two of its main ligands. F2-isoprostanes have been shown to mediate and promote fibrosis via TPr activation in hepatic stellate cells (HSC) from rats treated with cirrhosis-inducing carbon tetrachloride (Acquaviva 2013). HSCs play a significant role in the progression of cirrhosis by increasing the amount of extracellular matrix in the hepatic parenchyma.

In addition to the fibrotic processes, many of these same mediators have a more immediate effect on the blood flow through the hepatic sinusoids. TxA₂ serves as a vasoconstrictor, while nitric oxide vasodilates in the healthy liver (Yokoyama 2005). However, in the cirrhotic liver, studies in rats have revealed an increase in the hepatic production of TxA₂ which would then further increase portal hypertension via vasoconstriction (Graupera 2003; Rodriguez-Vilarruplas 2012). Also, the compensatory feedback between TxA₂ as a vasoconstrictor and nitric oxide as the inhibitory vasodilator seems to disappear in the cirrhotic liver (Yokoyama 2005).

Blockade of thromboxane-mediated signaling in the TPr has revealed an anti-fibrotic effect in two rat models of cirrhotic disease (Rosado 2013). Additionally, interrupting this signaling pathway lowered portal pressure suggesting a reduction in hepatic vascular resistance.

Clinical trials utilizing antagonists of other potent vasoconstrictors of the hepatic portal system (e.g. endothelin-1) have been completed with little success, suggesting that TxA₂ signaling may be of particular importance (Tripathi 2006). Therefore the development of a potential disease-modifying therapy in patients with portal hypertension would address a significant unmet medical need.

### SUMMARY OF THE INVENTION

It is an object of the present invention to provide new methods of preventing and/or treating portal hypertension in mammals, e.g., humans.

It is an object of the present invention to provide a composition and method for reducing the effects of portal hypertension in mammals, e.g., humans.

It is an object of the present invention to modify the progression of portal hypertension, as determined by hepatic venous pressure gradient (HVPG) measurement.

It is an object of the present invention to improve portal hypertension via treatment with a thromboxane A₂ receptor antagonist (e.g., ifetroban).

It is an object of the present invention to reduce portal hypertension in patients (e.g., as compared to placebo and e.g. as measured using a platelet function assessment by either rotational thromboelastometry or thromboelastography) via treatment with a thromboxane A₂ receptor antagonist (e.g., ifetroban).

It is an object of the present invention to improve portal hypertension in patients (e.g., as compared to placebo and, e.g., as measured by serum biomarkers for fibrosis and inflammation) via treatment with a thromboxane A₂ receptor antagonist (e.g., ifetroban).

It is an object of the present invention to reduce laboratory and physical evidence of portal hypertension in patients with cirrhosis (e.g. as compared to placebo and as measured e.g. by serum biomarkers, platelet function assessment and/or HVPG measurement).

It is an object of the present invention to improve quality of life in patients with portal hypertension.

In accordance with the above objects, the present invention provides compositions and methods for preventing, reversing, ameliorating or treating portal hypertension by administering a therapeutically effective amount of a thromboxane A₂ receptor antagonist (e.g., ifetroban or a pharmaceutically acceptable salt thereof, such as ifetroban sodium) to a patient in need thereof.

In certain preferred embodiments, the mammal is a human patient with portal hypertension and the therapeutically effective amount of the thromboxane A₂ receptor antagonist or a pharmaceutically acceptable salt thereof has an action selected from the group consisting of blockade of thromboxane-mediated signaling in the thromboxane receptor resulting in an anti-fibrotic effect in cirrhotic disease; interruption of the signaling pathway to lower portal pressure, thereby reducing hepatic vascular resistance and managing the symptoms of portal hypertension and combinations of any of the foregoing.

In certain preferred embodiments, the mammal is a human patient with portal hypertension and the therapeutically effective amount of the thromboxane A₂ receptor antagonist or a pharmaceutically acceptable salt thereof allows for short and/or long-term relief of portal hypertension and cirrhosis through reduction in vasoconstriction and fibrosis.

In certain embodiments, the present invention is directed to a method of treating and/or ameliorating portal hypertension, comprising administering to a patient, in need thereof, a therapeutically effective amount of a thromboxane A₂ receptor antagonist to provide a desired plasma concentration of the thromboxane A₂ receptor antagonist (and/or its active metabolites) of about 0.1 ng/ml to about 100,000 ng/ml. In certain embodiments, the therapeutically effective amount of a thromboxane A₂ receptor antagonist to provide a desired plasma concentration of the thromboxane A₂ receptor antagonist of about 0.1 ng/ml to about 10,000 ng/ml. In some embodiments, the afore-mentioned plasma concentration is a plasma concentration at a steady state. In some embodiments, the afore-mentioned plasma concentration is a maximum plasma concentration (Cmax). In certain preferred embodiments, the thromboxane A₂ receptor antagonist is ifetroban or a pharmaceutically acceptable salt thereof, e.g., ifetroban sodium.

In certain embodiments, the thromboxane A₂ receptor antagonist comprises a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]- benzenepropanoic acid (Ifetroban), and pharmaceutically acceptable salts thereof.

The invention is further directed to a method of treating portal hypertension in a mammal in need of treatment thereof, comprising administering a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (ifetroban), or a pharmaceutically acceptable salt thereof to the mammal. In certain embodiments, the thromboxane A₂ receptor antagonist comprises a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium). In certain preferred embodiments, the mammal is a human patient. In certain preferred embodiments, the therapeutically effective amount of ifetroban slows the progression of portal hypertension in the human patient and/or improves liver fibrosis.

In any of the methods described above and others described herein, the ifetroban is preferably administered in an amount effective to provide a plasma concentration of the ifetroban (and/or active metabolites of ifetroban) of about 1 ng/ml to about 100,000 ng/ml or of about 1 ng/ml to about 10,000 ng/ml for ifetroban itself, and in some embodiments from about 1 ng/ml to about 1,000 ng/ml. In some embodiments, the afore-mentioned plasma concentration is a plasma concentration at steady state. In some embodiments, the afore-mentioned plasma concentration is a maximum plasma concentration (Cmax). In certain preferred embodiments where the mammal is a human patient, the therapeutically effective amount is from about 100 mg to about 2000 mg per day, or from about 10 mg or about 100 mg to about 1000 mg per day, and certain embodiments more preferably from about 100 to about 500 mg per day. The daily dose may be administered in divided doses or in one bolus or unit dose or in multiple dosages administered concurrently. In this regard, the ifetroban may be administered orally, intranasally, rectally, vaginally, sublingually, buccally, parenterally, or transdermally.

In certain preferred embodiments, the pharmaceutical composition described above, the therapeutically effective amount is from about 10 mg to about 1000 mg ifetroban (or pharmaceutically acceptable salt thereof) per day. In certain preferred embodiments, the therapeutically effective amount is from about 100 mg to about 500 mg per day, and in certain embodiments from about 150 mg to about 350 mg per day.

The present invention also relates to methods and compositions for treating portal hypertension in a subject(s) or patient(s) in need of treatment thereof, particularly, the method comprising administering a therapeutically effective amount of a thromboxane A₂ receptor antagonist or, a pharmaceutically acceptable salt thereof, to a subject(s) or patient(s) in need thereof. In particular, it relates to a method of treating or preventing a disorder that results in portal hypertension, in a subject(s) or patient(s) in need of such treatment, comprising administering a composition comprising administering a therapeutically effective amount of a thromboxane A₂ receptor antagonist to a patient in need thereof in an amount effective to reduce vasoconstriction and/or fibrosis. Further provided is a method of preventing portal hypertension in a subject(s) or patient(s) in need of such treatment, comprising administering a composition comprising a thromboxane A₂ receptor antagonist in an amount effective to reduce the formation of fibrosis and/or vasoconstriction that would occur in the absence of such treatment.

The invention is further directed to a pharmaceutical composition comprising a thromboxane A₂ receptor antagonist or a pharmaceutically acceptable salt thereof, the thromboxane A₂ receptor antagonist being in an amount effective to treat a human patient with portal hypertension. In certain preferred embodiments, the thromboxane A₂ receptor antagonist is ifetroban or a pharmaceutically acceptable salt thereof. In certain preferred embodiments, the ifetroban salt is ifetroban sodium. In certain preferred embodiments, the therapeutically effective amount is from about 10 mg to about 1000 mg per day, and in certain embodiments from about 150 mg to about 350 mg. In other embodiments the therapeutically effective amount is about 250 mg per day. In certain preferred embodiments, the pharmaceutical composition is an oral solid dosage form.

The phrase “therapeutically effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

The term “thromboxane A₂ receptor antagonist” as used herein refers to a compound that inhibits the expression or activity of a thromboxane receptor by at least or at least about 30%, 35%, 40%, 45%, 50%, 55%, 60%, 65%, 70%, 75%, 80%, 85%, 90%, 95%, 96%, 97%, 98%, 99%, or 100% in a standard bioassay or in vivo or when used in a therapeutically effective dose. In certain embodiments, a thromboxane A₂ receptor antagonist inhibits binding of thromboxane A₂ to the receptor. Thromboxane A₂ receptor antagonists include competitive antagonists (i.e., antagonists that compete with an agonist for the receptor) and non-competitive antagonists. Thromboxane A₂ receptor antagonists include antibodies to the receptor. The antibodies may be monoclonal. They may be human or humanized antibodies. Thromboxane A₂ receptor antagonists also include thromboxane synthase inhibitors, as well as compounds that have both thromboxane A₂ receptor antagonist activity and thromboxane synthase inhibitor activity.

As used herein, the term “unit dose” refers to physically discrete units suitable as unitary dosages for mammalian subjects, each unit containing as the active ingredient a predetermined quantity of the thromboxane A₂ receptor antagonist.

The term “comprising” is an inclusive term interpreted to mean containing, embracing, covering or including the elements listed following the term, but not excluding other unrecited elements.

A “therapeutically effective amount” means the amount that, when administered to an animal for treating a disease, is sufficient to effect treatment for that disease.

As used herein, the term “treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “parenteral” as used herein, includes subcutaneous injections, intravenous, intramuscular, intrasternal injection or infusion techniques.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

### DETAILED DESCRIPTION OF THE INVENTION

In accordance with the above stated objects, it is believed that administration of a therapeutically effective amount of a thromboxane A₂ receptor antagonist to a subject(s) or patient(s) in need thereof can prevent and/or treat portal hypertension.

### Thromboxane A₂ Receptor Antagonist

The discovery and development of thromboxane A₂ receptor antagonists has been an objective of many pharmaceutical companies for approximately 30 years (see, Dogne J-M, et al., Exp. Opin. Ther. Patents 11: 1663-1675 (2001)). Certain individual compounds identified by these companies, either with or without concomitant thromboxane A₂ synthase inhibitory activity, include ifetroban (BMS), ridogrel (Janssen), terbogrel (BI), UK-147535 (Pfizer), GR 32191 (Glaxo), and S-18886 (Servier). Preclinical pharmacology has established that this class of compounds has effective antithrombotic activity obtained by inhibition of the thromboxane pathway. These compounds also prevent vasoconstriction induced by thromboxane A₂ and other prostanoids that act on the thromboxane A₂ receptor within the vascular bed, and thus may be beneficial for use in preventing and/or treating hepatorenal syndrome and/or hepatic encephalopathy.

Suitable thromboxane A₂ receptor antagonists for use in the present invention may include, for example, but are not limited to small molecules such as ifetroban (BMS; [1S-(1α,2α,3α,4α)]-2-[[3-[4-[(pentylamino)carbony-1]-2-oxazolyl]-7- oxabicyclo[2.2.1]hept-2 yl]methyl]benzenepropanoic acid), as well as others described in U.S. Patent Application Publication No. 2009/0012115, the disclosure of which is hereby incorporated by reference in its entirety.

Additional thromboxane A₂ receptor antagonists suitable for use herein are also described in U.S. Pat. No. 4,839,384 (Ogletree); U.S. Pat. No. 5,066,480 (Ogletree, et al.); U.S. Pat. No. 5,100,889 (Misra, et al.); U.S. Pat. No. 5,312,818 (Rubin, et al.); U.S. Pat. No. 5,399,725 (Poss, et al.); and U.S. Pat. No. 6,509,348 (Ogletree), the disclosures of which are hereby incorporated by reference in their entireties. These may include, but are not limited to, interphenylene 7-oxabicyclo-heptyl substituted heterocyclic amide prostaglandin analogs as disclosed in U.S. Pat. No. 5,100,889, including:

[1S-(1α,2α,3α,4α)]-2-[[3-[4-[[(4-cyclo-hexylbutyl)amino]carbonyl]-2-oxazolyl]-7-oxabicyclo[2.2.1]-hept-2-yl]methyl]benzenepropanoic acid (SQ 33,961), or esters or salts thereof;

[1S-(1α,2α,3α,4α)]-2-[[3-[4-[[[(4-chloro-phenyl)-butyl]amino]carbonyl]-2-oxazolyl]-7-oxabicyclo[2.2.1]hept-2-yl]methyl]benzenepropanoic acid or esters, or salts thereof;

[1S-(1α,2α,3α,4α)]-3-[[3-[4-[[(4-cycloh-exylbutyl)-amino]carbonyl]-2-oxazolyl]-7-oxabicyclo]2.2.1]hept-2-yl]benzene acetic acid, or esters or salts thereof;

[1S-(1α,2α,3α,4α)]-[2-[[3-[4-[[(4-cyclohexyl-butyl)amino]carbonyl]-2-oxazolyl]-7-oxabicyclo[2.2.1]hept-2-yl]methyl]phenoxy]acetic acid, or esters or salts thereof;

[1S-(1α,2α,3α,4α]-2-[[3-[4-[[(7,7-dime-thyloctyl)-amino]carbonyl]-2-oxazolyl]-7-oxabicyclo[2.2.1]hept-2-yl]-methyl]benzenepropanoic acid, or esters or salts thereof.

7-oxabicycloheptyl substituted heterocyclic amide prostaglandin analogs as disclosed in U.S. Pat. No. 5,100,889, issued Mar. 31, 1992, including [1S-[1α,2α(Z),3α,4α)]-6-[3-[4-[[(4-cyclohexylbutyl)amino]-carbonyl]-2-oxazolyl]- 7-oxabicyclo[2.2.1]hept-2-yl]-4-hexenoic acid, or esters or salts thereof;

[1S-[1α,2α(Z),3α,4α)]]-6-[3-[4-[[(4-cyclohexyl-butyl)amino]carbonyl]-2-thiazolyl]-7-[2.2.1]hept-2-yl]-4-hexenoic acid, or esters or salts thereof;

[1S-[1α,2α(Z),3α,4α)]]-6-[3-[4-[[(4-cyclohexyl-butyl)methylamino]carbonyl]-2-oxazolyl]-7-oxabicyclo-[2.2.1]hept-2-yl]- 4-hexenoic acid, or esters or salts thereof;

[1S-[1α,2α(Z),3α,4α)]]-6-[3-[4-[(1-pyrrolidinyl)-carbonyl]-2-oxazolyl]-7-oxabicyclo[2.2.1]hept-2-yl]-4-hexenoic acid, or esters or salts thereof;

[1S-[1α,2α(Z),3α,4α)]]-6-[3-[4-[(cyclohexylamino)-carbonyl]-2-oxazolyl]-7-oxabicyclo[2.2.1]hept-2-yl-4-hexenoic acid or esters or salts thereof;

[1S-[1α,2α(Z),3α,4α)]]-6-[3-[4-[[(2-cyclohexyl-ethyl)amino]carbonyl]-2-oxazolyl]-7-oxabicyclo[2.2.1]hept-2-yl]-4- hexenoic acid, or esters or salts thereof;

[1S-[1α,2α(Z),3α,4α)]]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof;

[1S-[1α,2α(Z),3α,4α)]-6-[3-[4-[[(4-chlorophenyl)-amino]carbonyl]-2-oxazolyl]-7-oxabicyclo[2.2.1]hept-2-yl]-4- hexenoic acid, or esters or salts thereof;

[1S-[1α,2α(Z),3α,4α)]]-6-[3-[4-[[[4-(4-chloro-phenyl)butyl]amino]carbonyl]-2-oxazolyl]-7-oxabicyclo-[2.2.1]hept-2-yl]-4-hexenoic acid, or esters or salts thereof;

[1S-[11α,2α(Z),3α,4α)]]-6-[3-[4.alpha.-[[-(6-cyclohexyl-hexyl)amino]carbonyl]-2-oxazolyl]-7-oxabicyclo[2.2.1]hept-2-yl]- 4-hexenoic acid, or esters, or salts thereof;

[1S-[1α,2α(Z),3α,4α)]]-6-[3-[4-[[(6-cyclohexyl-hexyl)amino]carbonyl]-2-oxazolyl]-7-oxabicyclo[2.2.1]hept-2-yl]-4- hexenoic acid, or esters or salts thereof;

[1S-[1α,2α(Z),3α,4α]]-6-[3-[4-[(propylamino)-carbonyl]-2-oxazolyl]-7-oxabicyclo[2.2.1]hept-2-yl]-4-hexenoic acid, or esters or salts thereof

[1S-[1α,2α(Z),3α,4α)]]-6-[3-[4-[[(4-butylphenyl)-amino]carbonyl]-2-oxazolyl]-7-oxabicyclo[2.2.1]hept-2-yl]-4- hexenoic acid, or esters or salts thereof;

[1S-[1α,2α(Z),3α,4α)]]-6-[3-[4-[(2,3-dihydro-1H-indol-1-yl)carbonyl]-2-oxazolyl]-7-oxabicyclo(2.2.1]hept-2-yl]-4-hexenoic acid, or esters or salts thereof;

[1S-[1α,2α(Z),3α,4α)]]-6-[3-[4-[[(4-cyclohexyl-butyl)amino]carbonyl]-2-oxazolyl]-7-oxabicyclo[2.2.1]hept-2-yl]-N- (phenylsulfonyl)-4-hexenamide;

[1S-[11α,2α(Z),3α,4α)]]-6-[3-[4-[[(4-cyclohexyl-butyl)amino]carbonyl]-2-oxazolyl]-N-(methylsulfonyl)-7-oxabicyclo[2-.2.1]hept-2-yl]-4-hexenamide;

[1S-[1α,2α(Z),3α,4α)]]-7-[3-[4-[[(4-cyclohexyl-butyl)amino]carbonyl]-2-oxazolyl]-7-oxabicyclo(2.2.1]hept-2-yl]-5- heptenoic acid, or esters or salts thereof;

[1S-[1α,2α(Z),3α,4α)]]-6-[3-[4-[[(4-cyclohexyl-butyl)amino]carbonyl]-1H-imidazol-2-yl]-7-oxabicyclo-[2.2.1]hept-2- yl]-4-hexenoic acid or esters or salts thereof;

[1S-[1α,2α,3α,4α)]-6-[3-[4-[[(7,7-dimethyloctyl)-amino]carbonyl]-2-oxazolyl]-7-oxabicyclo[2.2.1]hept-2-yl]-4-hexenoic acid, or esters or salts thereof;

[1S-[1α,2α(E),3α,4α)]]-6-[3-[4-[[(4-cyclohexyl-butyl)amino]carbonyl]-2-oxazolyl]-7-oxabicyclo[2.2.1]hept-2-yl]-4- hexenoic acid;

[1S-[1α,2α,3α,4α)]-3-[4-[[(4-(cyclohexylbutyl)-amino]carbonyl]-2-oxazolyl]-7-oxabicyclo[2.2.1]heptane-2-hexanoic acid or esters or salts thereof,

[1S-[1α,2α(Z),3α,4α)]]-6-[3-[4-[[(4-cyclohexyl-butyl)amino]carbonyl]-2-oxazolyl]-7-oxabicyclo-[2.2.1]hept-2-yl]-4- hexenoic acid, or esters or salts thereof;

7-oxabicycloheptane and 7-oxabicycloheptene compounds disclosed in U.S. Pat. No. 4,537,981 to Snitman et al, the disclosure of which is hereby incorporated by reference in its entirety, such as [1S-(1α,2α(Z),3α(1E,3S*,4R*),4α)]]-7-[3-(3-hydroxy-4-phenyl-1-pentenyl)-7-oxabicyclo[2.2.1]hept-2-yl]-5-heptenoic acid (SQ 29,548); the 7-oxabicycloheptane substituted aminoprostaglandin analogs disclosed in U.S. Pat. No. 4,416,896 to Nakane et al., the disclosure of which is hereby incorporated by reference in its entirety, such as [1S-[1α,2α(Z),3α,4α)]]-7-[3-[[2-(phenylamino)carbonyl]-hydrazino]methyl]-7- oxabicyclo[2.2.1]hept-2-yl]-5-heptenoic acid; the 7-oxabicycloheptane substituted diamide prostaglandin analogs disclosed in U.S. Pat. No. 4,663,336 to Nakane et al, the disclosure of which is hereby incorporated by reference in its entirety, such as, [1S-[1α,2α(Z),3α,4α)]]-7-[3-[[[[(1-oxoheptyl)amino]-acetyl]amino]methyl]-7-oxabicyclo[2.2.1]hept-2-yl]-5- heptenoic acid and the corresponding tetrazole, and [1S-[1α,2α(Z),3α,4α)]]-7-[3-[[[[(4-cyclohexyl-1-oxobutyl)-amino]acetyl]amino]methyl]-7-oxabicyclo]2.2.1]hept-2-yl]-5-heptenoic acid;

7-oxabicycloheptane imidazole prostaglandin analogs as disclosed in U.S. Pat. No. 4,977,174, the disclosure of which is hereby incorporated by reference in its entirety, such as [1S-[1α,2α(Z),3α,4α)]]-6-[3-[[4-(4-cyclohexyl-1- hydroxybutyl)-1H-imidazole-1-yl]methyl]-7-oxabicyclo[2.2.1]hept-2-yl]-4-hexenoic acid or its methyl ester;

[1S-[1α,2α(Z),3α,4α)]]-6-[3-[[4-(3-cyclohexyl-propyl)-1H-imidazol-1-yl]methyl]-7-oxabicyclo[2.2.1]hept-2-yl]-4- hexenoic acid or its methyl ester;

[1S-[1α,2α(X(Z),3α,4α)]]-6-[3-[[4-(4-cyclohexyl-1-oxobutyl)-1H-imidazol-1-yl]methyl]-7-oxabicyclo[2.2.1]hept-2- yl]-4-hexenoic acid or its methyl ester;

[1S-[1α,2α(Z),3α,4α]]-6-[3-(1H-imidazol-1-ylmethyl)-7-oxabicyclo[2.2.1]hept-2-yl]-4-hexenoic acid or its methyl ester; or

[1S-[1α,2α(Z),3α,4α)]]-6-[3-[[4-[[(4-cyclohexyl-butyl)amino]carbonyl]-1H-imidazol-1-yl]methyl-7-oxabicyclo-[2.2.1]-hept- 2-yl]-4-hexenoic acid, or its methyl ester;

The phenoxyalkyl carboxylic acids disclosed in U.S. Pat. No. 4,258,058 to Witte et al, the disclosure of which is hereby incorporated by reference in its entirety, including 4-[2-(benzenesulfamido)ethyl]phenoxy-acetic acid (BM 13,177-Boehringer Mannheim), the sulphonamidophenyl carboxylic acids disclosed in U.S. Pat. No. 4,443,477 to Witte et al, the disclosure of which is hereby incorporated by reference in its entirety, including 4-[2-(4-chlorobenzenesulfonamido)ethyl]-phenylacetic acid (BM 13,505, Boehringer Mannheim), the arylthioalkylphenyl carboxylic acids disclosed in U.S. Pat. No. 4,752,616, the disclosure of which is hereby incorporated by reference in its entirety, including 4-(3-((4-chlorophenyl)sulfonyl)propyl)benzene acetic acid.

Other examples of thromboxane A₂ receptor antagonists suitable for use herein include, but are not limited to vapiprost (which is a preferred example), (E)-5-[[[(pyridinyl)]3-(trifluoromethyl)phenyl]methylene]amino]-oxy]pentanoic acid also referred to as R68,070-Janssen Research Laboratories, 3-[1-(4-chlorophenylmethyl)-5-fluoro-3-methylindol-2-yl]-2,-2-dimethylpropanoic acid [L-655240 Merck-Frosst) Eur. J. Pharmacol. 135(2):193, Mar. 17, 1987], 5(Z)-7-([2,4,5-cis]-4-(2-hydroxyphenyl)-2-trifl-uoromethyl-1,3-dioxan-5-yl)heptenoic acid (ICI 185282, Brit. J. Pharmacol. 90 (Proc. Suppl):228 P-Abs, March 1987), 5(Z)-7-[2,2-dimethyl-4-phenyl-1,3-dioxan-cis-5-yl]heptenoic acid (ICI 159995, Brit. J. Pharmacol. 86 (Proc. Suppl):808 P-Abs., December 1985), N,N′-bis[7-(3-chlorobenzeneamino-sulfony-l)-1,2,3,4-tetrahydro-isoquinolyl]disulfonylimide (SKF 88046, Pharmacologist 25(3):116 Abs., 117 Abs, August 1983), (1.alpha.(Z)-2.beta.,5.alpha.]-(+)-7-[5-[[(1,1′-biphenyl)-4-yl]-methoxy]-2-(4-morpholinyl)-3-oxocyclopentyl]-4-heptenoic acid (AH 23848-Glaxo, Circulation 72(6):1208, December 1985, levallorphan allyl bromide (CM 32,191 Sanofi, Life Sci. 31 (20-21):2261, Nov. 15, 1982), (Z,2-endo-3-oxo)-7-(3-acetyl-2-bicyclo[2.2.1]heptyl-5- hepta-3Z-enoic acid, 4-phenyl-thiosemicarbazone (EP092-Univ. Edinburgh, Brit. J. Pharmacol. 84(3):595, March 1985); GR 32,191 (Vapiprost)-[1R-[1.alpha.(Z),2.beta.,3.beta.,5.alpha.]]-(+)-7-[5-([1,1′-biphenyl]-4-ylmethoxy)-3-hydroxy-2-(1- piperidinyl)cyclopentyl]-4-heptenoic acid; ICI 192,605-4(Z)-6-[(2,4,5-cis)2-(2-chlorophenyl)-4-(2-hydroxyphenyl)-1,3-dioxan-5-yl]hexenoicacid; BAY u 3405 (ramatroban)-3-[[(4-fluorophenyl)-sulfonyl]amino]-1,2,3,4-tetrahydro-9H-c-arbazole-9-propanoic acid; or ONO 3708-7-[2.alpha.,4.alpha.-(dimethylmethano)-6.beta.-(2-cyclopentyl-2.beta.-hydroxyacetami-do)-1.alpha.-cyclohexyl]-5(Z)-heptenoic acid; (.+−.)(5Z)-7-[3-endo-((phenylsulfonyl)amino]-bicyclo[2.2.1]hept-2-exo-yl]-heptenoic acid (S-1452, Shionogi domitroban, Anboxan®.); (−)6,8-difluoro-9-p-methylsulfonylben-zyl-1,2,3,4-tetrahydrocarbazol-1-yl-acetic acid (L670596, Merck) and (3-[1-(4-chlorobenzyl)-5-fluoro-3-methyl-indol-2-yl]-2,2-dimethylpropanoic acid (L655240, Merck).

The preferred thromboxane A₂ receptor antagonist of the present invention is ifetroban or any pharmaceutically acceptable salts thereof.

In certain preferred embodiments the preferred thromboxane A₂ receptor antagonist is ifetroban sodium (known chemically as [1S-(1α,2α,3α,4α)]-2-[[3-[4-[(Pentylamino)carbonyl]-2-oxazolyl]-7-oxabicyclo[2.2.1]hept-2-yl]methyl]- benzenepropanoic acid, monosodium salt.

### Methods of Treatment

In certain embodiments of the present invention there is provided a method of preventing and/or treating and/or ameliorating portal hypertension in a patient or patient population by administration of a therapeutically effective amount of a thromboxane A₂ receptor antagonist to a patient(s) in need thereof.

The administration of a therapeutically effective amount of a thromboxane A₂ receptor antagonist may be accomplished via any therapeutically useful route of administration, including but not limited to orally, intranasally, rectally, vaginally, sublingually, buccally, parenterally, or transdermally. In certain preferred embodiments, the thromboxane A₂ receptor antagonist is administered parenterally. In certain further embodiments, the thromboxane A₂ receptor antagonist is administered by intra-articular injection. In certain further embodiments, the thromboxane A₂ receptor antagonist is administered directly to the affected anatomic site. In another embodiment, the thromboxane A₂ receptor antagonist is administered through the hepatic artery.

In any of the methods described above and others described herein, the thromboxane A₂ receptor antagonist (e.g., ifetroban) is preferably administered in an amount effective to provide a plasma concentration of the thromboxane A₂ receptor antagonist (and/or active metabolites thereof) of about 1 ng/ml to about 100,000 ng/ml or of about about 0.1 ng/ml; or 1 ng/ml to about 10,000 ng/ml for ifetroban itself, and in some embodiments from about 1 ng/ml to about 1,000 ng/ml or more (e.g., in some embodiments up to about 10,000 ng/ml, and in further embodiments up to about 100,000 ng/ml). In some embodiments, the afore-mentioned plasma concentration is a plasma concentration at steady state. In some embodiments, the afore-mentioned plasma concentration is a maximum plasma concentration (Cmax). In certain preferred embodiments where the mammal is a human patient, the therapeutically effective amount is from about 100 mg to about 2000 mg per day, or from about 10 mg or about 100 mg to about 1000 mg per day, and certain embodiments more preferably from about 100 to about 500 mg per day. The daily dose may be administered in divided doses or in one bolus or unit dose or in multiple dosages administered concurrently.

In this regard, the ifetroban may be administered orally, intranasally, rectally, vaginally, sublingually, buccally, parenterally, or transdermally.

In one embodiment where the mammal is a human patient, the therapeutically effect amount of ifetroban is about 250 mg daily, taken orally.

The dose administered should be adjusted according to age, weight and condition of the patient, as well as the route of administration, dosage form and regimen and the desired result.

In order to obtain the desired plasma concentration of thromboxane A₂ receptor antagonists for the treatment or prevention of portal hypertension, daily doses of the thromboxane A₂ receptor antagonists preferably range from about 0.1 mg to about 5000 mg. In certain preferred embodiments, the daily dose of thromboxane A₂ receptor antagonists for the treatment or prevention of portal hypertension may range from about 1 mg to about 2000 mg; about 10 mg to about 1000 mg; from about 100 mg to about 1000 mg; from about 50 mg to about 500 mg; about 100 mg to about 500 mg; or from about 150 mg to about 300 mg per day.

In certain preferred embodiments, a daily dose of ifetroban sodium from about 10 mg to about 500 mg, preferably from about 150 mg to about 300 mg (ifetroban free acid amounts) will produce therapeutically effective plasma levels of ifetroban free acid for the treatment or prevention of portal hypertension.

When the thromboxane A₂ receptor antagonist is ifetroban, the desired plasma concentration should be greater than about 10 ng/mL (ifetroban free acid). The dose administered must be carefully adjusted according to age, weight and condition of the patient, as well as the route of administration, dosage form and regimen and the desired result.

In certain preferred embodiments where the thromboxane A₂ receptor antagonist is ifetroban or a pharmaceutically acceptable salt thereof, a daily dose of ifetroban sodium from about 10 mg to about 500 mg, preferably from about 150 mg to about 300 mg (ifetroban free acid amounts) will produce effective plasma levels of ifetroban free acid.

### Pharmaceutical Compositions

The thromboxane A₂ receptor antagonists of the present invention may be administered by any pharmaceutically effective route. For example, the thromboxane A₂ receptor antagonists may be formulated in a manner such that they can be administered orally, intranasally, rectally, vaginally, sublingually, buccally, parenterally, or transdermally, and thus, be formulated accordingly.

In certain embodiments, the thromboxane A₂ receptor antagonists may be formulated in a pharmaceutically acceptable oral dosage form. Oral dosage forms may include, but are not limited to, oral solid dosage forms and oral liquid dosage forms.

Oral solid dosage forms may include, but are not limited to, tablets, capsules, caplets, powders, pellets, multiparticulates, beads, spheres and any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

Depending on the desired release profile, the oral solid dosage forms of the present invention may contain a suitable amount of controlled-release agents, extended-release agents, modified-release agents.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol and combinations thereof.

In certain embodiments of the present invention, the thromboxane A₂ receptor antagonists may be formulated into a dosage form suitable for parenteral use. For example, the dosage form may be a lyophilized powder, a solution, suspension (e.g., depot suspension).

In other embodiments, the thromboxane A₂ receptor antagonists may be formulated into a topical dosage form such as, but not limited to, a patch, a gel, a paste, a cream, an emulsion, liniment, balm, lotion, and ointment.

### DETAILED DESCRIPTION OF THE PREFERRED EMBODIMENTS

The following examples are not meant to be limiting and represent certain embodiments of the present invention.

### Example 1

In this example, ifetroban sodium capsules are prepared with the following ingredients listed in Table 1:

TABLE 1

| Ingredients | Percent by weight |
|---|---|
| Na salt of Ifetroban | 35 |
| Mannitol | 50 |
| Microcrystalline Cellulose | 8 |
| Crospovidone | 3.0 |
| Magnesium Oxide | 2.0 |
| Magnesium Stearate | 1.5 |
| Colloidal Silica | 0.3 |

The sodium salt of ifetroban, magnesium oxide, mannitol, microcrystalline cellulose, and crospovidone is mixed together for about 2 to about 10 minutes employing a suitable mixer. The resulting mixture is passed through a #12 to #40 mesh size screen. Thereafter, magnesium stearate and colloidal silica are added and mixing is continued for about 1 to about 3 minutes.

The resulting homogeneous mixture is then compressed into tablets each containing 35 mg, ifetroban sodium salt.

### Example II

In this example, 1000 tablets each containing 400 mg of ifetroban sodium are produced from the following ingredients listed in Table 2:

TABLE 2

| Ingredients | Amount | |
|---|---|---|
| Na salt of Ifetroban | 400 | gm |
| Corn Starch | 50 | g |
| Gelatin | 7.5 | g |
| Microcrystalline Cellulose (Avicel) | 25 | g |
| Magnesium Stearate | 2.5 | g |

### Example III

In this example. An injectable solution of ifetroban sodium is prepared for intravenous use with the following ingredients listed in Tables 3a and 3b:

TABLE 3a

| Ingredients | Amount | |
|---|---|---|
| Ifetroban Sodium | 2500 | mg |
| Methyl Paraben | 5 | mg |
| Propyl Paraben | 1 | mg |
| Sodium Chloride | 25,000 | mg |
| Water for injection q.s. | 5 | liter |

TABLE 3b

| Ingredients | Amount | |
|---|---|---|
| Ifetroban Sodium | 52.5 | mg mg |
| Sodium Phosphate Dibasic anhydrous | 345 | mg |
| Sodium Phosphate Monobasic Anhydrous | 1 | mg |
| Sodium Chloride | 21.5 | mg |
| Water for injection q.s. | 5 | liter |

The sodium salt of ifetroban, buffers and sodium chloride are dissolved in 3 liters of water for injection and then the volume is brought up to 5 liters. The solution is filtered through a sterile filter and aseptically filled into pre-sterilized vials which are then closed with pre-sterilized rubber closures. Each vial contains a concentration of 50 mg of active ingredient per 5 ml of solution.

### Example IV

A phase 2 multicenter, double-blind, randomized, double-blind, placebo-controlled study in patients to assess the safety and efficacy of ifetroban for the treatment of portal hypertension in cirrhotic patients. There will be a 90 day blinded treatment in this study with a 7-day screening period, a 90-day treatment period where blinded investigational treatment is given as an initial intravenous (IV) dose, then daily doses of oral IMP. A 7-day follow up completes the study. Thirty cirrhotic patients with portal hypertension will be enrolled in this study, with 20 receiving ifetroban and 10 receiving placebo. Diagnosis and main criteria for inclusion are:
1. Cirrhosis defined by histology or historical HVPG>7 mm Hg, or confirmed by liver stiffness measurement (LSM) above diagnostic threshold (15 kPa for Vibration Controlled Transient Elastography (VCTE) or 6.7 kPa for Magnetic Resonance Elastography (MRE) AND evidence of splenomegaly or collaterals OR platelet count below 150×103 with AST>ALT;
2. At least two stable baseline values for AST, ALT, ALP and bilirubin taken at screening and between 15 and 90 days prior. Stable is defined as values having a difference<40 U/L for AST, ALT and ALP and <3.0 mg/dL for total serum bilirubin;
3. Baseline hepatic venous pressure gradient between 8 and 16 mmHg, inclusive.
   Main criteria for exclusion are:
1. Less than 18 or more than 70 years of age;
2. Portal or splenic thrombosis;
3. Transjugular intrahepatic shunt (TIPS);
4. Active GI/variceal hemorrhage within the last 60 days;
5. Hemodialysis;
6. Child-Pugh score>12, calculated within 30 days of enrollment;
7. MELD-Na score>20;
8. Platelet count<80×103/μL;
9. History of bleeding diathesis or risk factors based on patient or familial history, other than cirrhosis and its sequelae;
10. Current acute kidney injury (AKI), chronic kidney disease (CKD) or hepatorenal syndrome (HRS) or a baseline SCr>2.0 mg/dL;
11. MI in the 90 days prior to enrollment;
12. Current need for endothelin receptor antagonists, somatostatin analogues or prostanoids, treatment for viral hepatitis, anticoagulant or antiplatelet drugs. Investigational Medicinal Product (IMP), dose and mode of administration.

Subjects will be administered a loading IV infusion of 150 mg ifetroban or placebo, followed by 250 mg ifetroban or placebo taken orally each day for 90 days.

The drug product is supplied as a capsule dosage form (size #1, white opaque) for oral administration. The formulation consists of ifetroban, mannitol, microcrystalline cellulose, crospovidone, magnesium oxide, colloidal silicon dioxide, and magnesium stearate. Capsules are filled into high density polyethylene bottles and sealed with screw-cap closures. Placebo for ifetroban capsules are formulated as a dry powder blend filled into capsules. The formulation consists of microcrystalline cellulose, crospovidone, colloidal silicon dioxide, and magnesium stearate. Capsules are filled into high density polyethylene bottles and sealed with screw-cap closures. Ifetroban and placebo capsules should be administered in a fasting state. Meals following administration should be held for at least 30 minutes following study drug administration.

The objectives of the study are as follows:

To evaluate the following in cirrhotic patients with portal hypertension who are treated with ifetroban:
Safety
Portal pressure
Serum liver enzymes
Surrogate markers of liver fibrosis and inflammation
Occurrence of variceal bleeds

### CONCLUSION

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification is to be regarded in an illustrative manner rather than a restrictive sense.

### REFERENCES

Acquaviva A, Vecchio D, Arezzini B, et al. 2013. Signaling pathways involved in isoprostane-mediated fibrogenic effects in rat hepatic stellate cells. Free Rad Biol Med. 65:201-7.
Buck M, Garcia-Tsao G, Groszmann R J, et al. Novel inflammatory biomarkers of portal pressure in compensated cirrhosis patients. Hepatology. 2014; 59(3):1052-9.
Deng Y Q, Zhao H, Ma A L, et al. 2015. Selected cytokines serve as potential biomarkers for predicting liver inflammation and fibrosis in chronic hepatitis B patients with normal to mildly elevated aminotransferases. Medicine. 94(45): doi 10.1097.
Graupera M, Gracia-Pagan, Pares M, et al. 2003. Cyclooxygenase-1 inhibition corrects endothelial dysfunction in cirrhotic rat livers. J Hepatology. 39: 515-21.
LaVecchia C, Levi F, Lucchini F, et al. 1994. Worldwide patterns and trends in mortality from liver cirrhosis, 1955 to 1990. Annals of Epidemiology. 4(6):480-6.
Pagliaro L, D'Amico G, Malizia G, et al. Portal hypertension in cirrhosis; natural history. In: Bosch J, Broszmann R (eds) Portal hypertension: Pathophysiology and treatment. Blackwell Scientific, Cambridge: 72-92.
Rodriguez-Vilarruplas A, Lavina B, Garcia-Caldero H, et. al. 2012. PPARa activation improves endothelial dysfunction and reduces fibrosis and portal pressure in cirrhotic rats. J of Hepatology. 56: 1033-9
Rosado E, Rodríguez-Vilarrupla A, Gracia-Sancho J, et al. 2013. Terutroban, a TP-receptor antagonist, reduces portal pressure in cirrhotic rats. Hepatology. 00(00):1-12.
Snyder N, Gajula L, Xiao S Y. 2006. APRI: an easy and validated predictor of hepatic fibrosis in chronic hepatitis C. J Clin Gastroenterol. 40(6):535-42.
Tripathi, D, Therapondos G, Ferguson J W, et al. 2006. Endothelin-1 contributes to maintenance of systemic but not portal haemodynamics in patients with early cirrhosis: a randomised controlled trial. Gut. 55:1290-5.
Yokoyama Y, Nimura Y, Nagino M, et al. 2005. Role of thromboxane in producing hepatic injury during hepatic stress. Arch Surg. 140: 801-7.

What is claimed is:

1. A method of treating or ameliorating portal hypertension in a mammal in need of treatment thereof, comprising administering a therapeutically effective amount of a thromboxane A₂ receptor antagonist or a pharmaceutically acceptable salt thereof to the mammal.

2. The method of claim 1, wherein the mammal is a human patient with cirrhosis.

3. The method of claim 2, wherein the thromboxane A₂ receptor antagonist is (1α,2α,3α,4α)]-2-[[3-[4-[(Pentylamino)carbonyl]-2-oxazolyl]-7-oxabicyclo[2.2.1]hept-2-yl]methyl]-benzenepropanoic acid (ifetroban), or a pharmaceutically acceptable salt thereof to the mammal.

4. The method of claim 3, wherein the therapeutically effective amount of ifetroban reduces the rate of formation of liver fibrosis and/or vasoconstriction in the mammal.

5. The method of claim 3, wherein the ifetroban is administered in an amount effective to provide a plasma concentration of the ifetroban of about 1 ng/ml to about 10,000 ng/ml.

6. The method of claim 3, wherein the thromboxane A₂ receptor antagonist is administered in an amount effective to provide a plasma concentration from about 1 ng/ml to about 100,000 ng/ml.

7. The method of claim 3, wherein the therapeutically effective amount is from about 10 mg to about 1000 mg per day.

8. The method of claim 7, wherein the ifetroban is administered orally, intranasally, rectally, vaginally, sublingually, buccally, parenterally, or transdermally.

9. The method of claim 7, wherein the mammal is a human patient and the therapeutically effective amount of ifetroban slows the progression of portal hypertension in the patient.

10. The method of claim 7, wherein the mammal is a human patient and the therapeutically effective amount of ifetroban reduces hepatic vascular resistance in the patient.

11. The method of claim 7, wherein the mammal is a human cirrhosis patient and the therapeutically effective amount of thromboxane A₂ receptor antagonist modifies the progression of portal hypertension.

12. The method of claim 7, wherein the mammal is a human patient and the therapeutically effective amount of ifetroban improves liver fibrosis and/or vasoconstriction in the patient.

13. The method of claim 7, wherein the mammal is a human patient and the therapeutically effective amount of ifetroban reduces portal hypertension in the patient compared to placebo as measured by a test selected from the group consisting of Hepatic Venous Pressure Gradient Measurement, platelet function assessment, e.g. by rotational thromboelastometry, thromboelastography, and evaluation of serum biomarkers for fibrosis and inflammation and combinations of any of the foregoing.

14. The method of claim 7, wherein the mammal is a human patient and the therapeutically effective amount of ifetroban improves liver function.

16. The method of claim 7, wherein the mammal is a human cirrhosis patient and the therapeutically effective amount of ifetroban improves laboratory and physical evidence of portal hypertension in the patient compared to placebo as measured by a test selected from the group consisting of Hepatic Venous Pressure Gradient Measurement, platelet function assessment, e.g. by rotational thromboelastometry, thromboelastography, and test of serum biomarkers for fibrosis and inflammation and combinations of any of the foregoing.

17. The method of claim 3 wherein the therapeutically effective amount is from about 150 mg to about 350 mg per day and the ifetroban is administered orally.

18. The method of claim 1, wherein the mammal is a human patient with portal hypertension and the therapeutically effective amount of the thromboxane A₂ receptor antagonist or a pharmaceutically acceptable salt thereof has an action selected from the group consisting of blockade of thromboxane-mediated signaling in the thromboxane receptor resulting in an anti-fibrotic effect in cirrhotic disease, interruption of the signaling pathway to lower portal pressure, thereby reducing hepatic vascular resistance and managing the symptoms of portal hypertension and combinations of any of the foregoing.

19. The method of claim 3, wherein the therapeutically effective amount is about 250 mg per day.

20. The method of claim 1, wherein the mammal is a human patient with portal hypertension and the therapeutically effective amount of the thromboxane A₂ receptor antagonist or a pharmaceutically acceptable salt thereof allows for short and/or long-term relief of portal hypertension and cirrhosis through reduction in vasoconstriction and fibrosis.
